(19) United States
(12) Patent Application Publication
(10) Pub. No.: US 2019/0159919 A1
(43) Pub. Date: May 30, 2019

(54) ARTICULATED ROD OF AN ORTHOPEDIC DEVICE

(71) Applicant: ORTHOSERVICE AG, Chiasso (CH)

(72) Inventors: Francesco TURCONI, Monza (MB) (IT); Daniele ALBERIO, Imbersago (LC) (IT)

(21) Appl. No.: 16/200,840

(22) Filed: Nov. 27, 2018

(30) Foreign Application Priority Data
Nov. 29, 2017 (IT) 102017000136919

(51) Int. Cl. A61F 5/01

(52) U.S. Cl. CPC A61F 5/0102; A61F 2005/0165; A61F 2005/0158

(57) ABSTRACT

Articulated rod of an orthopedic device including a pair of arms, two cover plates, a polycentric joint that articulates the pair of arms in flexion-extension rotation, at least one insert that constrains a rotation width, at least one stop pin for locking the at least one insert, a pair of half covers slidably associated through the guide means for passing from an open position to a closed position where at least one half cover keeps the stop pin in position by interference.

### CROSS REFERENCE TO RELATED APPLICATION

This application is related to and claims the benefit of Italian Patent Application Number 102017000136919 filed on Nov. 29, 2017, the entire contents of which are incorporated herein by reference.

### TECHNICAL FIELD

The present disclosure refers to an articulated rod of an orthopedic device.

### BACKGROUND

In the state of the art there are articulated rods of orthopedic devices with polycentric joints that can be adjusted in flexion-extension comprising inserts for constraining at least a flexion or extension rotation within a rotation width. The insert is associated with the polycentric joint through the use of screws that make the mounting and replacement step of the inserts on the orthopedic device disadvantageously complex.

The presence of threaded holes and screws makes it difficult for the orthopedic personnel needing to arrange suitable tools to apply the inserts.

Other locking elements of the inserts disadvantageously increase the size of the articulated rod which should instead be small and as compact as possible since it is an element of the orthopedic device.

### BRIEF SUMMARY

The disclosure provides an articulated rod in which the inserts are stably maintained in position during the use of an orthopedic device, the inserts can be easily replaced for adjusting an extension-flexion rotation width and the articulated rod is compact, stable and small.

### BRIEF DESCRIPTION OF THE DRAWINGS

The characteristics and advantages of an articulated rod with a polycentric joint that can be adjusted in extension-flexion according to the present disclosure will be more apparent from the following description, which is to be understood as exemplifying and not limiting, with reference to the schematic attached drawings, wherein:

FIG. 1 shows a partially exploded perspective view of an articulated rod of an orthopedic device with a polycentric joint;

FIG. 2 shows an exploded front perspective view of the articulated rod comprising a guide base, two half covers and the locking bar;

FIG. 3 shows an exploded rear perspective view of the articulated rod;

FIG. 4 shows a perspective view from below of the locking bar;

FIG. 5 shows a perspective view from above of a guide base;

FIG. 6 shows a perspective view from below of the guide base;

FIG. 7 shows a perspective view from below of two half covers;

FIG. 8 shows a plan view from below of an open position of the two half covers mounted with the guide base and with the locking bar;

FIG. 9 shows a plan view from below of a closed position of the two half covers mounted with the guide base and with the locking bar.

### DETAILED DESCRIPTION

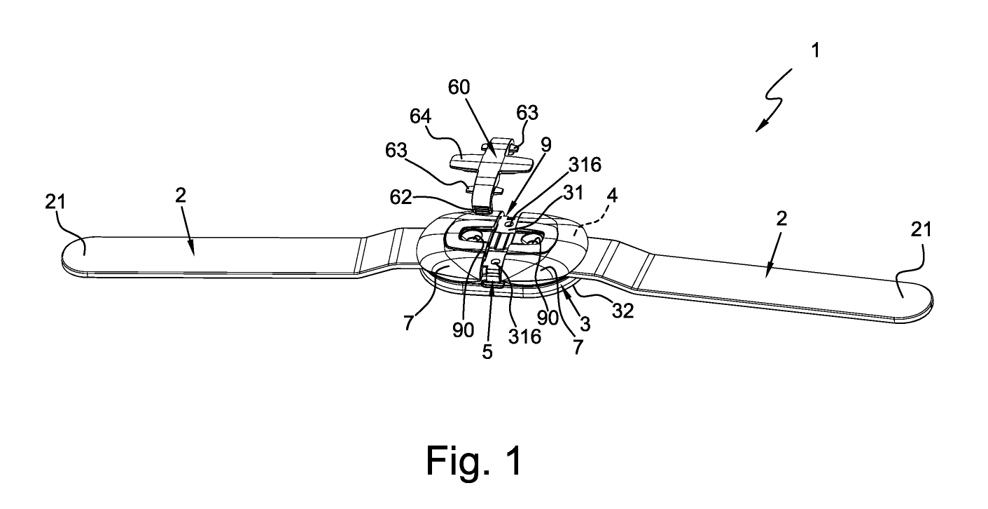

With particular reference to FIGS. 1-3, an articulated rod 1 of an orthopedic device is shown comprising a pair of arms 2, two cover plates 3 and a polycentric joint 4 that is mounted with the two cover plates 3 and articulates the pair of arms 2.

The two cover plates 3 are an outer cover plate 31 and an inner cover plate 32, where the terms inner and outer refer to mounting directions with respect to the orthopedic device.

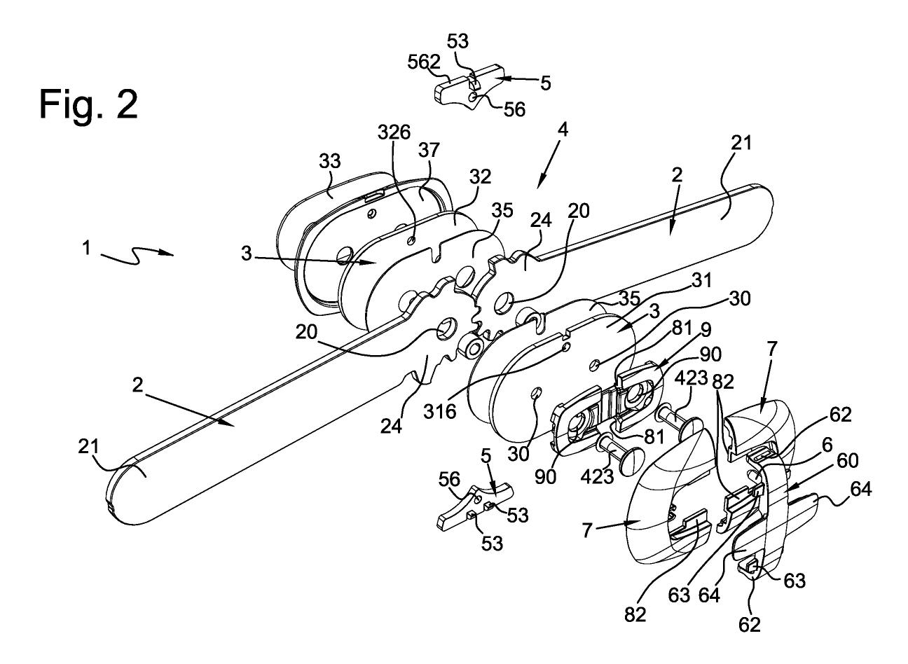

As shown in FIG. 2, the two cover plates 31, 32 mount a gasket 35 for promoting the movement of the polycentric joint 4. The inner cover plate 32 envisages the mounting of an inner cover 37 comprising edges that match with the pair of half covers 7. Towards the inside the inner cover 37 mounts a piece of Velcro 33.

In general the polycentric joint 4 allows a mutual rotation of the pair of arms 2. The rotation comprises two rotation directions which are a direction of flexion rotation and a direction of extension rotation.

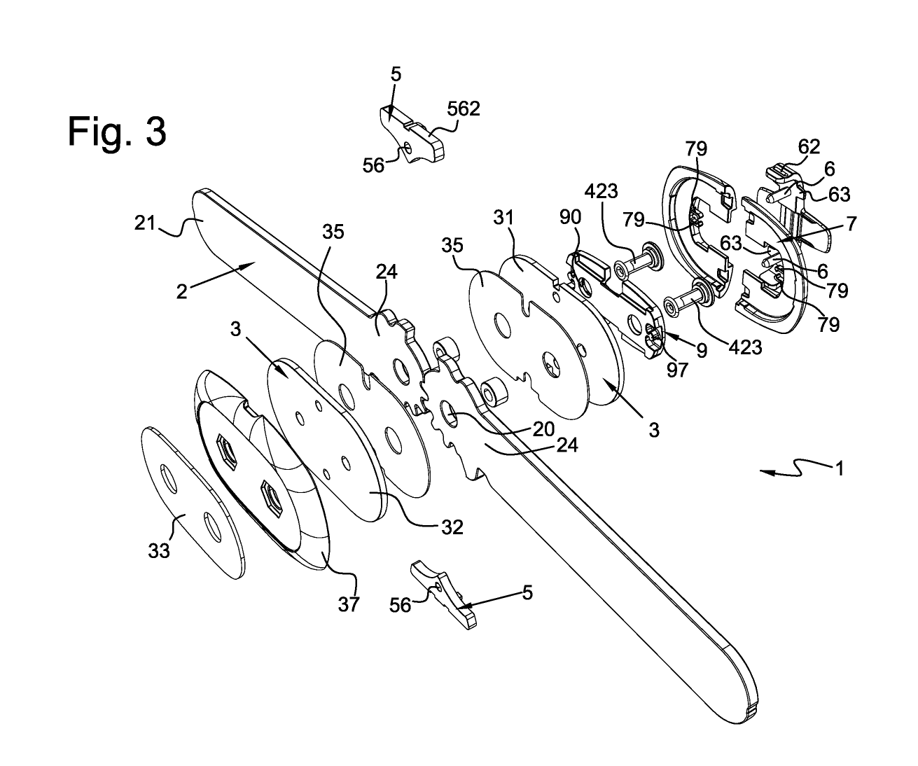

In the embodiment shown in particular in FIG. 3 each arm 2 bears a first end 21 that can be associated with the orthopedic device and a toothed end 24 opposite the first end 21.

The toothed ends 24 of the pair of arms 2 are meshed together for performing the rotation on a geometric plane of rotation coplanar thereto. Each toothed end 24 bears a central hole 20 in the axial position with respect to the extension of the toothed profile.

The two cover plates 3 are coupled to each other to form a shell and house the two toothed ends 24 and two rotation pins 423. The two rotation pins 423 are arranged orthogonally with respect to the geometric plane of rotation. The two pins are respectively engaged in central holes 20 of the arms 2 and in the through holes 30 of the two cover plates 3.

As shown in FIG. 3, the articulated rod 1 further comprises two removable inserts 5. Each insert 5 is selectively engageable in a volume comprised between the two plates 3 and the arms 2.

The flexion-extension rotation width of the pair of arms 2 is not constrained by the insert 5 when the insert 5 is not engaged in the polycentric joint 4.

FIG. 3 shows an example of the insert 5 that has a wedge shape for the engagement of the two arms 2. Projections 53 of the insert 5 allow the preliminary centering of the insert 5 with respect to the plate 31. Inserts 5 of different shapes or sizes may envisage allowing a plurality of flexion and extension rotation widths of the two arms 2. It follows that the flexion rotation width may also be different from the extension rotation width.

The articulated rod 1 also comprises two stop pins 6, i.e. one stop pin 6 for every insert 5. Each stop pin 6 is engageable in the respective insert 5 and with at least one of the cover plates 3.

Even more advantageously, as in the example described in FIGS. 1-9, it is possible to envisage each stop pin 6 engaging the insert 5 with both of the cover plates 3 so as to make the engagement even more secure.

The stop pin 6 has a longitudinal dimension that extends along an orthogonal direction to the geometric plane of rotation.

To place the stop pin 6 in position, the insert 5 bears a through hole 56, the outer cover plate 31 bears a through hole 316, the inner cover plate 32 bears a through hole 326, where such through holes 56, 316 and 326 are aligned with each other so as to be engaged by the stop pin 6 in position. The through holes 56, 316 and 326 are not threaded and the stop pin 6 is not a screw, advantageously simplifying the block.

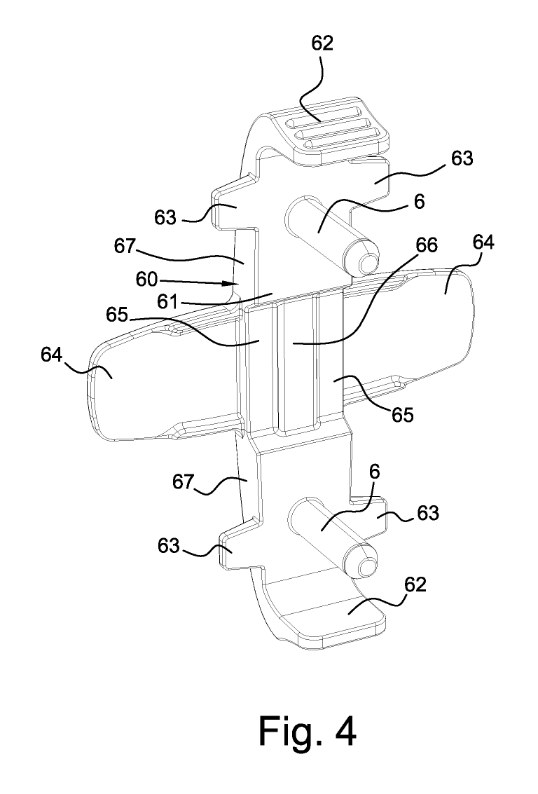

Even more advantageously, the two stop pins 6 can simultaneously engage the two inserts 5 with at least one of the two plates 3, and it is therefore envisaged that the two stop pins 6 are a single piece with a locking bar 60, as shown in FIG. 4, or that the two stop pins 6 are mounted solidly with the locking bar 60, an operation that would not be possible to actuate with locking screws of the state of the art as they need to be screwed separately. Mounting the two stop pins 6 on a single locking bar 60 allows the inserts 5 locking operation to be quicker, more secure and easier.

As shown in particular in FIG. 4, the locking bar 60 comprises a base 61 that rests on a geometric plane parallel to the geometric plane of rotation.

From the base 61 the two stop pins 6 are parallel to each other and project orthogonally to the geometric plane of the base 61.

The locking bar 60 comprises at the ends thereof two walls 62 substantially orthogonal to the base 61 that extend for a height sufficient to at least partially cover a lateral thickness 562 of the insert 5 so that each wall 62 is a containing element and provides greater security for maintaining the insert 5 even more firmly in position in the polycentric joint 4.

Each wall 62 has a prehensile portion for making it advantageously easy to position and remove the locking bar 60 from its own seat.

The locking bar 60 further comprises two pairs of fins 63. Each fin 63 is an extension of the base 61 and lies on a geometric plane parallel to the geometric plane of the base 61. In the embodiment shown in particular in FIG. 4, the geometric plane on which the fins 63 lie is the same geometric plane on which the base 61 of the locking bar 60 lies, but these two geometric planes may also be at different heights.

Each pair of fins 63 is advantageously arranged in proximity to the respective stop pin 6 so as to reduce the clearances of the locking bar 60 and allow the positioning of the stop pins 6 more securely in the holes 56, 316, 326.

According to the embodiment shown in the figures, the locking bar 60 also comprises a pair of covers 64 that cover the two rotation pins 423.

The locking bar 60 comprises interconnection elements that are grooves 65 and a step 66, which are arranged in the central part of the locking bar 60.

The locking bar 60 has side walls 67.

As shown in FIGS. 2, 3, 5, 6, the articulated rod 1 comprises a guide base 9 that is integral with the outer cover plate 31.

The guide base 9 comprises two through holes 90 at the central holes 20 and the through holes 30. The two through holes 90 are engaged by the two rotation pins 423 that make the guide base 9 integral with the outer cover plate 31, as shown in FIGS. 1-3.

According to a further embodiment, not shown in the figures, the guide base 9 could be made as a single piece with the outer cover plate 31, or completely absent.

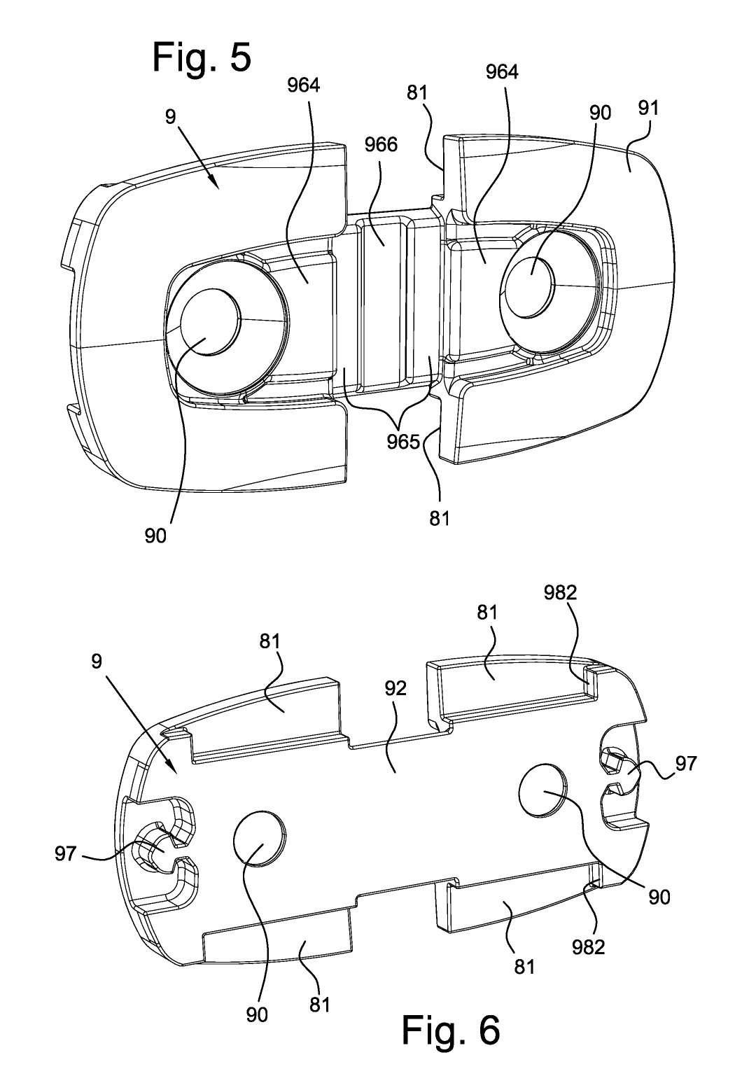

The guide base 9 comprises an outer face 91, shown in FIG. 5, and an inner face 92, shown in FIG. 6. The outer face 91 comes into contact with the locking bar 60. The inner face 92 is in contact with the outer cover plate 31.

The guide base 9 comprises interconnection elements which are steps 965 and a groove 966. In particular, the steps 965 and the grooves 966 are provided on the outer face 91.

A coupling between the grooves 65 of the locking bar 60 and the steps 965 of the guide base 9 and a related coupling between the step 66 of the locking bar 60 and the grooves 966 of the guide base 9 allow the locking bar 60 to be advantageously positioned more securely and without any clearance and therefore the stop pins 6 to be positioned more securely and the inserts 5 to be kept more stably positioned without any clearances.

Alternatively it is possible to envisage the steps 965 and the groove 966 being in a single piece with the outer cover plate 31.

The outer face 91 of the guide base 9 bears a pair of grooves 964 adapted to house the pair of covers 64 of the locking bar 60 so that the pair of covers 64 advantageously covers the through holes 90 of the guide base 9. Even more advantageously the thickness of the covers 64 corresponds to the height of the grooves 964 so that the outer surface of the articulated rod 1 is advantageously smooth and free from projections or grooves that could get caught in the use of the orthopedic device.

As shown in particular in FIGS. 2-3, 5-9, the articulated rod 1 comprises a pair of half covers 7 and guide means 81, 82 coupled to each other for slidably associating the pair of half covers 7.

As shown in particular in FIGS. 5 and 6, the guide base 9 bears a first element 81 of the guide means and each of the half covers 7 bears a second element 82 of the guide means. In this way, the two half covers 7 are slidably associated with the guide base 9.

With particular reference to FIG. 6, the first element 81 of the guide means comprises two pairs of grooves 81 of the guide base 9. The inner face 92 bears a first pair of grooves 81 arranged at one end of the guide base 9 for guiding a first half cover 7 and a second pair of grooves 81 arranged at an opposite end of the guide base 9 for guiding a second half cover 7.

As shown in particular in FIG. 6, the guide base 9 also comprises two pairs of stroke end stops 982, i.e. a pair of stroke end stops 982 for each end of the guide base 9, so that each of the half covers 7 can slide on the first element 81 of the guide means from a closed position of the half cover 7 to an open position of the half cover 7, being it constrained to the guide base 9 by the stroke end stops 982.

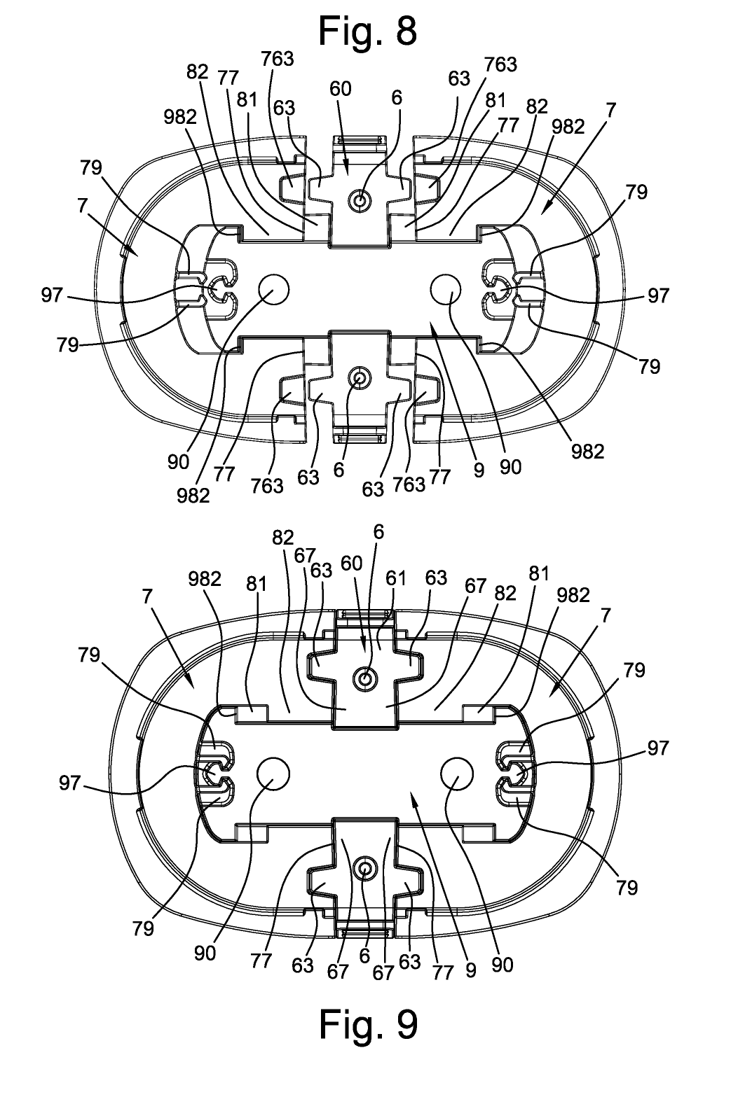

As shown in particular in FIG. 8, the open position of the half cover 7 envisages that the half cover 7 can slide open until it abuts with the pair of stroke end stops 982 of the guide base 9.

As shown in particular in FIG. 9, the closed position of the half cover 7 envisages that the half cover 7 can slide closed until it abuts with the respective side wall 67 of the locking bar 60.

As shown in particular in FIG. 6, the guide base 9 bears a pair of interlocking elements 97 for the respective engagement elements 79 of the half covers 7. Each end of the guide base 9 bears the interlocking element 97. Each half cover 7 bears the engagement element 79 adapted to fit with the respective interlocking element 97 of the guide base 9 so as to advantageously secure the half cover 7 with the guide base 9.

Alternatively it is possible to envisage that each end of the guide base 9 can bear a plurality of interlocking elements 97 and that a respective plurality of engagement elements 79 of the half covers 7 is provided.

As shown in particular in FIGS. 6, 8 and 9, the interlocking element 97 is a mushroom-shaped extension.

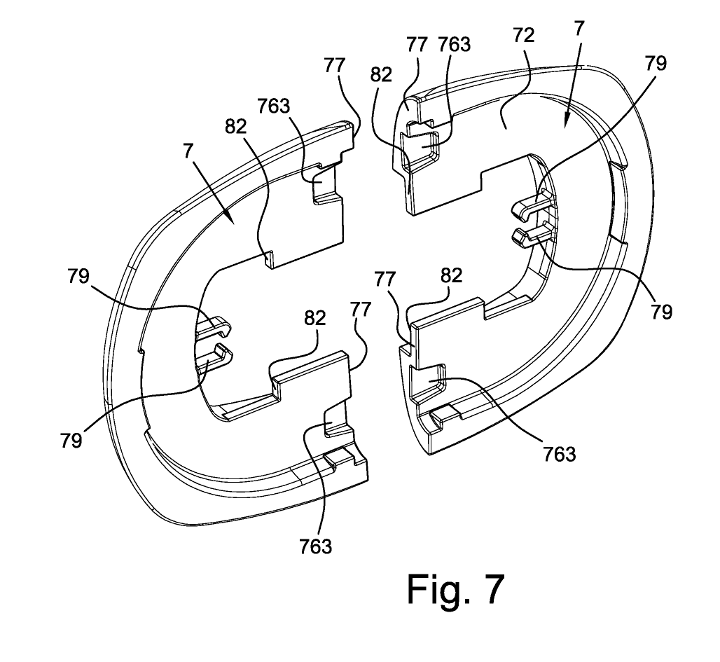

As shown in particular in FIGS. 7, 8 and 9, the engagement element 79 of the half cover 7 comprises two elastic extensions that engage the mushroom shape of the respective interlocking element 97 of the guide base 9.

As shown in particular in FIGS. 2-3 and 7-9, each half cover 7 of the pair of half covers 7 bears the second element 82 of the guide means.

The second element 82 of the guide means is an extension of the half cover 7 arranged on a geometric plane at a different height with respect to the geometric plane on which the first element 81 of the guide means is arranged, so that the first element 81 acts as a guide for the sliding of the second element 82 of the guide means.

The half covers 7 pass from a closed position interfering with the stop pins 6 to an open position free from interference with the stop pins 6.

The closed position of the half cover 7 envisages that at least one element 763 of the half cover 7 enters into interference with at least one portion 63 that is integral with the at least one stop pin 6 locking the at least one stop pin 6 in engagement with the polycentric joint 4.

The open position of the half covers 7 envisages that no element of the half cover 7 enters into interference with any portion of the at least one stop pin 6, allowing the removal of the at least one stop pin 6 from the polycentric joint 4.

As shown in particular in FIGS. 7-9, each half cover 7 has an inner face 72 that bears two grooves 763 at the respective pairs of fins 63 of the locking bar 60, so that once the two half covers 7 are in the closed position, as shown in FIG. 9, the fins 63 are locked by the grooves 763 of the half covers 7, as the fins 63 are covered by the grooves 763 preventing the removal, which may even be accidental, of the stop pin 6 from the polycentric joint 4. When the half covers 7 are in the closed position, the coupling between the grooves 763 and the fins 63 advantageously allows the removal of the stop pins 6 from the polycentric joint 4 to be prevented.

Even more advantageously it is envisaged that the grooves 763 are arranged at an abutment portion 77 of the two half covers 7, so that when the half covers 7 are in the closed position the abutment portion 77 abuts against side walls 67 of the locking bar 60 so as not to leave any gaps and the outer surface of the articulated rod is smooth, so as not to get caught during use of the orthopedic device.

In relation to the adjustment of the articulated rod 1 by the orthopedic personnel based on the specific needs of the patient, with the half covers in the open position, the inserts 5 are positioned in the polycentric joint 4. Subsequently, the locking bar 60 inserts the stop pins 6 into the through holes 316, 56 and 326 locking the inserts 5 in the polycentric joint 4. As shown in FIGS. 8 and 9, the two half covers 7 are made to pass from the open position to the closed position ensuring that the engagement and interlocking elements 79 and 97 block the mutual movement of the two half covers 7 firmly and securely maintaining the stop pins 6 and the respective inserts 5 in the polycentric joint 4.

Advantageously the articulated rod 1, according to the disclosure, has inserts 5 that are easily replaceable to adjust the flexion-extension rotation width, hence being easily and quickly mountable, secure, compact and small.

Alternatively the outer face 91 bears the first element 81 of the guide means. The second element 82 of the guide means has a complementary shape to the first element 81, so as to allow the sliding of the half covers 7 with the guide base 9.

Alternatively the outer cover plate 31 bears the first element 81 of the guide means. In this alternative the half covers 7 are slidably associated with the outer cover 31.

Even more alternatively it is possible to envisage that only one of the two half covers 7 is slidably associated with the guide base 9 and the other half cover 7 remains integral with the guide base 9.

Alternatively it is envisaged that the half covers 7 comprise an interlocking element and a respective engagement element so that the two half covers 7 can be secured directly to each other in the closed position, e.g. by interlocking, rather than being secured to the guide base 9. In this case it is envisaged that the half covers 7 cover at least one portion of the stop pins 6 so that the stop pins 6 cannot exit from the through holes 56, 316, 326, the at least one portion of the stop pins 6 being in interference by abutment with at least one portion of the half covers 7. For example, it is predictable that the half covers 7 in the closed position cover the locking bar 60 fully or partly to prevent it exiting its seat.

Alternatively it is sufficient that only one half cover 7 of the pair of half covers 7 is adapted to pass from the closed position to the open position. In this alternative the closed position envisages that at least one element 763 of one of the two half covers 7 enters into interference with at least one portion 63 of the at least one stop pin 6 blocking the at least one stop pin 6 or both of the stop pins 6. The articulated rod 1 according to the disclosure can be applied indifferently to orthopedic devices for the upper or lower limbs.

The articulated rod thus conceived is susceptible to many modifications and variations, all falling within the disclosure; furthermore, all the details are replaceable by technically equivalent elements. In practice, the materials used, as well as the dimensions, can be any according to the technical requirements.

1. Articulated rod of an orthopedic device comprising
a pair of arms,
two cover plates,
a polycentric joint articulating said pair of arms in mutual rotation, where said rotation comprises a flexion rotation direction and an extension rotation direction, at least one removable insert, selectively engageable so as to block said flexion and/or extension rotation within a predetermined angular width,
at least one stop pin adapted to be in position axially engaging at least one through hole of said at least one insert and at least one through hole of at least one of the two cover plates, a pair of half covers and guide means coupled to each other in order to slidably associate at least one half cover of said pair of half covers, where said guide means comprise a first element integral with said at least one cover plate and a second element integral with said at least one half cover, said pair of half covers being adapted to pass from an open position to a closed position, where said closed position provides for at least one element of said at least one half cover to enter into interference with at least one portion of said at least one stop pin holding in position said at least one stop pin with respect to the translation along its own axis.

2. Articulated rod according to claim 1, further comprising a guide base which is integral with said at least one cover plate, said guide base bears said first element of the guide means which has a complementary shape with respect to said second element of the guide means so as to allow the sliding of said at least one half cover with said guide base.

3. Articulated rod according to claim 1, further comprising a locking bar which bears two mutually parallel stop pins, adapted to engage two inserts simultaneously.

4. Articulated rod according to claim 3, wherein said locking bar comprises, at its ends, two walls extended for a height sufficient to at least partially cover a lateral thickness of said at least one insert.

5. Articulated rod according to claim 3, wherein said at least one element of said at least one half cover is at least one groove of said at least one half cover and that said at least one portion of said at least one stop pin is at least one fin of said locking bar, where the closed position of said at least one half cover provides for said at least one fin to be covered by said at least one groove.

6. Articulated rod according to claim 3, wherein said guide base comprises interconnection elements adapted to be coupled with respective interconnection elements of the locking bar when said at least one stop pin is positioned.

7. Articulated rod according to claim 1, wherein the closed position of said pair of half covers provides for each half cover to be secured to the other half cover of said pair of half covers.

8. Articulated rod according to claim 2, wherein the closed position of said pair of half covers provides for each half cover of said pair of half covers to be secured with said guide base.

9. Articulated rod according to claim 8, wherein said guide base bears at least one interlocking element for each half cover of said pair of half covers and that each half cover bears at least one engagement element, where the closed position of the pair of half covers provides for said at least one engagement element to be adapted to engage with said interlocking element of the guide base.

10. Articulated rod according to claim 9, wherein the interlocking element of the guide base has a mushroom shape and that the engagement element of the half cover comprises two elastic extensions, where the closed position of the pair of half covers provides for said two elastic extensions to engage the mushroom shape of the respective interlocking element of the guide base.
